(19) United States
(12) Patent Application Publication
(10) Pub. No.: US 2006/0129130 A1
(43) Pub. Date: Jun. 15, 2006

(54) Sheath/catheter system with controlled hardness and flexibility

(76) Inventors: Michael G. Tal, Woodbridge, CT (US); Janelle R. Anderson, New York, NY (US)

Correspondence Address: WELSH & FLAXMAN LLC, 2000 DUKE STREET, SUITE 100, ALEXANDRIA, VA, 22314, US

(21) Appl. No.: 11/280,856

(22) Filed: Nov. 17, 2005

Related U.S. Application Data
(60) Provisional application No. 60/629,502, filed on Nov. 18, 2004.

(51) Int. Cl. A61M 25/00

(52) U.S. Cl. 604/525

(57) ABSTRACT

A sheath/catheter system includes a catheter including a proximal end and a distal end, the distal end including a distal tip portion exhibiting controlled hardness and flexibility. The system also includes a sheath including a proximal end and a distal end. The system further includes a mechanism for controlling the hardness and flexibility of the distal tip portion of the catheter. A method for vascular access is achieved by introducing a catheter into a vessel, advancing a sheath over the catheter and into the vessel, altering the catheter or sheath to change the respective flexibility and hardness thereof and further advancing the catheter or sheath within the vascular system to a predetermined location.

### CROSS REFERENCE TO RELATED APPLICATION

This application is based upon U.S. Provisional Patent Application Ser. No. 60/629,502, entitled “VASCULAR SHEATH AND ANGIOGRAPHIC CATHETER WITH VARIABLE STIFFNESS”, filed Nov. 18, 2004.

### BACKGROUND OF THE INVENTION

1. Field of the Invention

The invention relates to a method and apparatus adapted for vascular access. More particularly, the present invention relates to a sheath/catheter system providing for enhanced flexibility and softness for utilization and access through tortuous vessels. In addition, the invention relates to a method for utilizing the present sheath/catheter system.

2. Description of the Prior Art

A variety of techniques have been developed for the delivery of expandable grafts and/or stents for use within blood vessels or ducts for repairing blood vessels narrowed or occluded by disease. As those skilled in the art will appreciate, stents are commonly implanted within blood vessels, arteries or veins to maintain or restore the patency of the passageway. Stents are commonly deployed percutaneously to minimize the invasiveness of the procedure.

Percutaneous deployment is initiated by access into the vascular system of the patient, typically into the femoral artery. A tubular or sheath portion of an introducer is inserted through the incision and extends into the artery. The introducer has a central lumen that provides a passageway through the patient's skin and artery wall into the interior of the artery. A hub and valve body portion of the introducer remains outside the patient's body to prevent blood from leaking out of the artery along the outside of the sheath. A distal end of a guide wire is passed through the introducer passageway into the patient's vasculature. The guide wire is threaded through the vasculature until it reaches the treatment sight. In carotid artery interventions, the sheath is long enough to extend from the patient's groin to the carotid artery. A sheath is placed in the carotid artery for injection of a contrast medium to visualize the area to be treated and to allow exchanges of various devices such as balloons, stents and embolic capturing devices for safe and efficient stenting procedures.

More specifically, and with reference to FIGS. 1a to 1i, the method of advancing the sheath into the carotid artery is currently performed in the following manner. First, the carotid artery is selected with a soft tip, shaped catheter. Thereafter, a relatively floppy guide wire is inserted into the vessel. A floppy guide wire is utilized as a stiffer guide wire will not make the turns and will push the catheter out of the vessel. A soft catheter is then advanced over the soft guide wire and into the target vessel. The soft guide wire is then removed from the catheter. The catheter is then flushed to prevent blood clot formation on its tip. A stiffer guide wire is then inserted into the catheter and into the carotid artery.

It should be noted that a stiff guide wire is more likely to cause scraping of the vessel wall and cause embolic complications. This is an important consideration since there is no embolic protection device in place at this stage of the procedure. Access to the carotid artery has to be achieved before the target lesion can be safely crossed and the embolic protection device can be placed.

The tip of the guide wire is sometimes advanced into the external carotid artery to prevent inadvertent advancement into the internal carotid during guide wire exchanges. The stiff wire straightens the system and allows subsequent advancement of the sheath.

Once the stiffer guide wire is in position, the soft catheter is removed and the access sheath is advanced over the stiffer wire and into the carotid artery. Once the sheath is in place, the dilator and stiffer wire are removed. Finally, a floppy tip catheter and a thin (0.018-0.014 inch) guide wire are inserted into the sheath for safe crossing of the area to be treated.

As those skilled in the art appreciate, this is a complicated multi-step technique and requires multiple guide wire and catheter exchanges. The many exchanges may cause debris dislodgment during the procedure and result in a stroke.

The many exchanges are a result of the fact that currently used catheters and sheaths have a fixed stiffness. It is known that when catheterizing a tortuous vessel, a softer catheter is more easily advanced over a guide wire. However, these soft sheaths or catheters do not provide sufficient support for advancement of a device to the area requiring treatment. As a result, and in view of the stiffness limitations of catheters and sheaths currently available, advancing and positioning of a stiff sheath in a tortuous vessel requires multiple steps as discussed above. This multi-step technique with multiple wiring catheter exchanges often causes trauma to vessel walls and inadvertent dislodgment of plaque may lead to embolization and stroke.

With the foregoing in mind, those skilled in the art will appreciate that improved access instruments are currently required. The present invention attempts to overcome the shortcomings of prior art devices and procedures by providing a sheath/catheter system with adjustable stiffness. In addition, the present invention attempts to provide a method for utilizing the present sheath/catheter system in the carotid artery or any target vessel with fewer steps and no exchanges.

### SUMMARY OF THE INVENTION

It is, therefore, an object of the present invention to provide a sheath/catheter system. The system includes a catheter including a proximal end and a distal end, the distal end including a distal tip portion exhibiting controlled hardness and flexibility. The system also includes a sheath including a proximal end and a distal end. The system further includes a mechanism for controlling the hardness and flexibility of the distal tip portion of the catheter.

It is also an object of the present invention to provide a method for vascular access. The method is achieved by introducing a catheter into a vessel, advancing a sheath over the catheter and into the vessel, altering the catheter or sheath to change the respective hardness and flexibility thereof and further advancing the catheter or sheath within the vascular system to a predetermined location.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIGS. 1a through 1i show the prior art technique for carotid artery access.

FIGS. 2a through 2e show a carotid artery access technique in accordance with the present invention.

FIG. 3 is a schematic of a sheath/catheter system in accordance with the present invention.

FIG. 4 is a cross sectional view of a sheath in accordance with the embodiment disclosed with reference to FIG. 3.

FIGS. 5 and 6 are cross sectional views showing alternate embodiments of a sheath utilized in conjunction with the system disclosed in FIG. 3.

FIG. 7 is a cross sectional view of a catheter utilized in conjunction with the sheath/catheter system shown in FIG. 3.

FIGS. 8 and 9 are cross sectional views of alternate embodiments of a catheter for use in conjunction with the sheath/catheter system shown in FIG. 3.

FIG. 10 is an alternate embodiment of a sheath/catheter system wherein the sheath does not include a metal coil.

FIG. 11 is an alternate embodiment of a sheath/catheter system in accordance with the present invention.

FIG. 12 is a cross sectional view of a sheath for use in conjunction with the sheath/catheter system disclosed with reference to FIG. 11.

FIGS. 13 and 14 are respectively cross sectional views along the lines XIII-XIII and XIV-XIV of the sheath shown in FIG. 12.

FIG. 15 shows an alternate fluid lumen construction in accordance with the present invention.

FIG. 16 is a cross sectional view a catheter for use in conjunction with the sheath/catheter system disclosed with reference to FIG. 11.

FIGS. 17 and 18 are respectively cross sectional views along the lines XVII-XVII and XVIII-XVIII of the catheter shown in FIG. 16.

FIG. 19 shows an alternate fluid lumen construction in accordance with the present invention.

FIG. 20 is an alternate embodiment of the sheath/catheter system wherein the sheath is constructed without the fluid lumen.

FIGS. 21 and 22 are cross sectional views respectively showing a sheath and lumen in accordance with an alternate embodiment.

FIGS. 23 and 24 are cross sectional views respectively showing a sheath and catheter employing insulating material in accordance with an alternate embodiment.

FIGS. 25 to 28 show various catheter tip designs for use in accordance with the present invention.

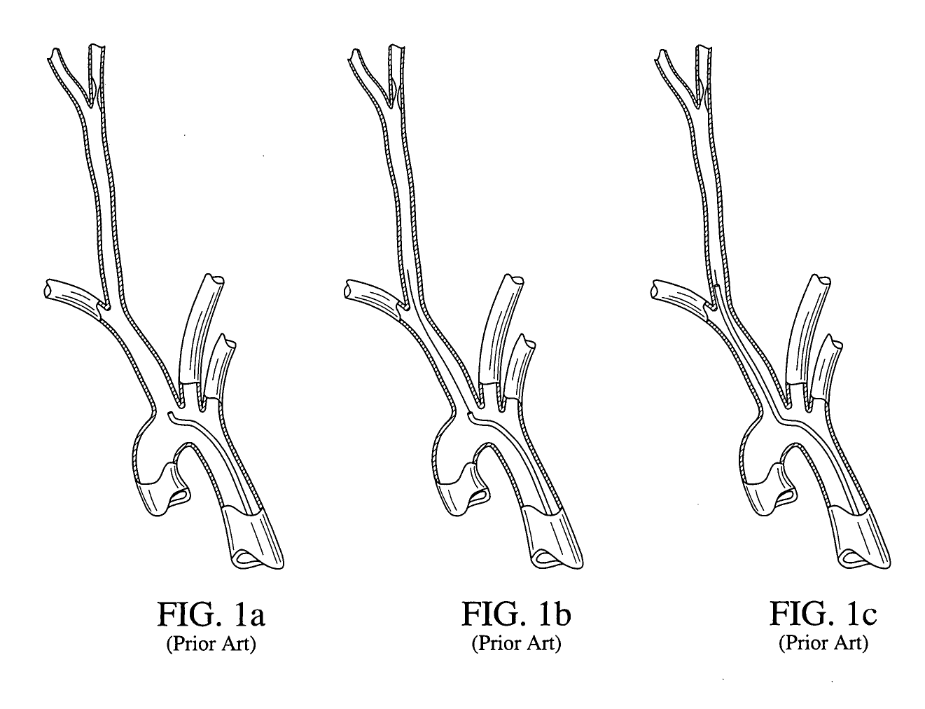

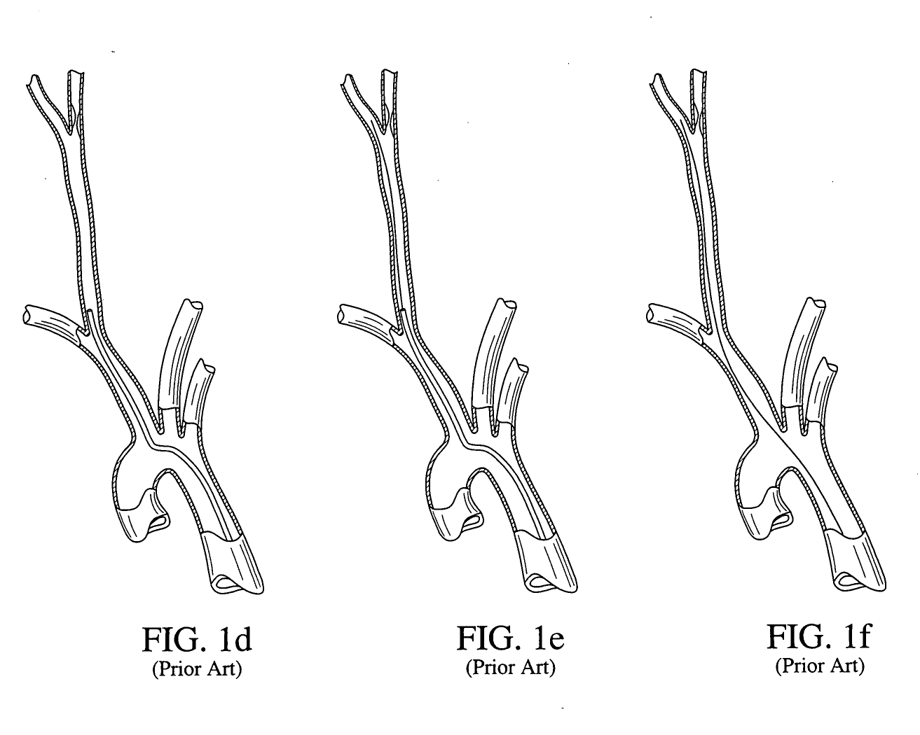

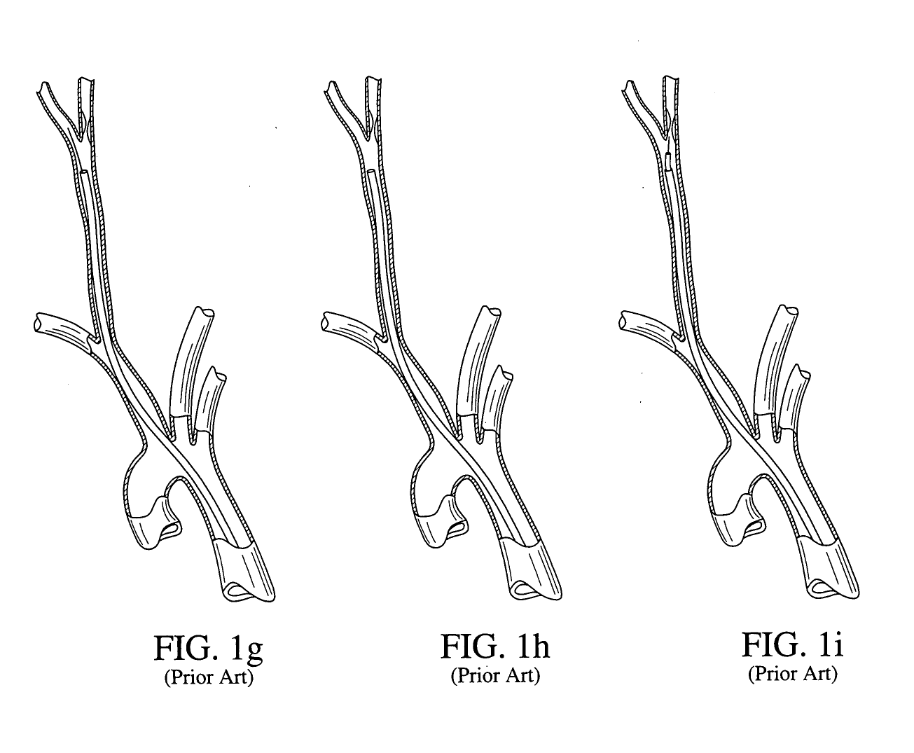

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as the basis for the claims and as a basis for teaching one skilled in the art how to make and/or use the invention.

Referring to the various figures and embodiments presented in accordance with the present invention, the present invention relates to a method and apparatus for improving vascular access. In particular, the invention relates to a vascular sheath/catheter system specifically for use in access to the carotid arteries. Improved access to the carotid arteries is achieved by providing a sheath/catheter system that offers a selectively changeable stiffness and/or hardness under the control of the medical practitioner performing the procedure.

Terms relating to the stiffness and hardness of the sheath/catheter system are used throughout the present disclosure. With that in mind, those skilled in the art will appreciate that terms such as soft, hard, etc. relate to the ability of a surface to compress (for example, like a hard or soft pillow), while terms such as stiff, flexibility, etc. relate to an article's ability to bend about a longitudinal axis (for example, like a stiff or flexible fishing pole). In accordance with a preferred embodiment hardness and flexibility are changed at the same time, although it is contemplated that it may be desirable to distinctly alter the hardness and flexibility of the sheath/catheter system. With this in mind, these terms relate to relative measures and the object of the present invention to selectively change the hardness and/or flexibility of a sheath and/or catheter for improving vascular access.

The stiffness/softness is altered based upon the needs of the particular patient and the specific anatomy of the patient. The stiffness/softness of the sheath/catheter system may be changed before, during or after insertion into the patient. In addition to the sheath/catheter system disclosed in accordance with the present invention, a novel method for carotid artery access is also disclosed.

Although the present invention is disclosed herein with reference to carotid artery procedures, those skilled in the art will appreciate the present invention may be utilized in conjunction with a variety of vascular procedures without departing from the spirit of the present invention. For example, the present invention could be utilized in conjunction with vascular access procedures requiring a long sheath to travel through a tortuous anatomy to a particular vessel prior to angioplasty or stenting. With this in mind, it is contemplated the present invention may be applicable to numerous interventional neuroradiology applications. The present invention is also contemplated as being applicable to endovascular procedures requiring a catheter to be advanced over a guide wire into a vessel at an angle. It is often difficult to advance a stiff catheter over a wire and into a branch positioned at an angle. With this in mind, the present invention may be applied in achieving access to the contralateral iliac and femoral arteries over the aortic bifurcation for access to arteries of the contralateral lower extremities.

As the following disclosure will make clear, the present invention provides a replacement for currently used sheaths and catheters, particularly, those sheaths and catheters used in conjunction with carotid access. The present invention allows for use of sheaths and catheters for safer access with reduced trauma to the vessels en route to the final position. The present invention also reduces the risk of embolic complications.

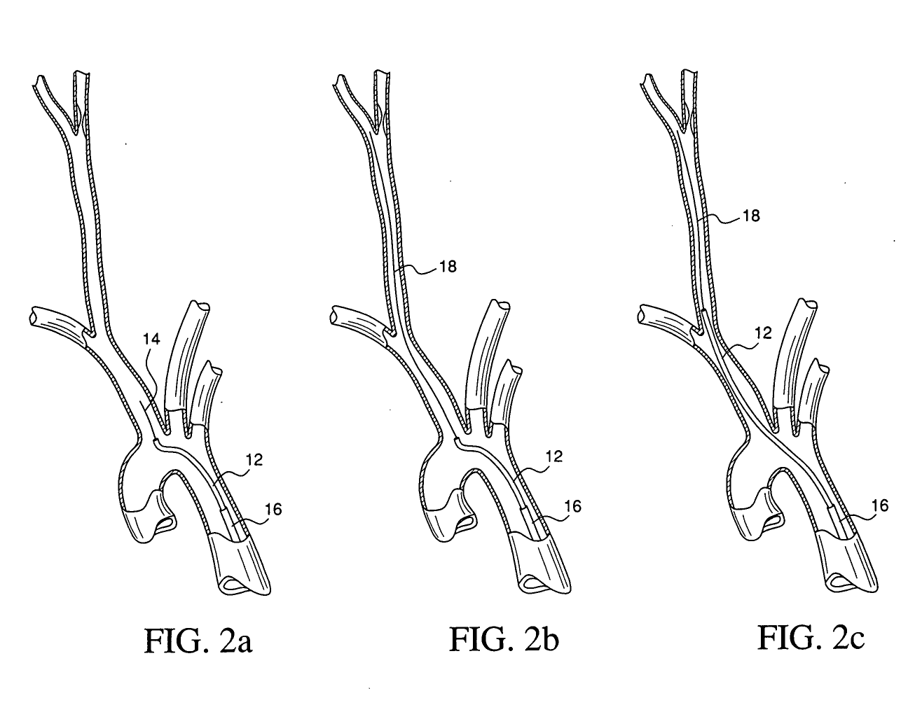

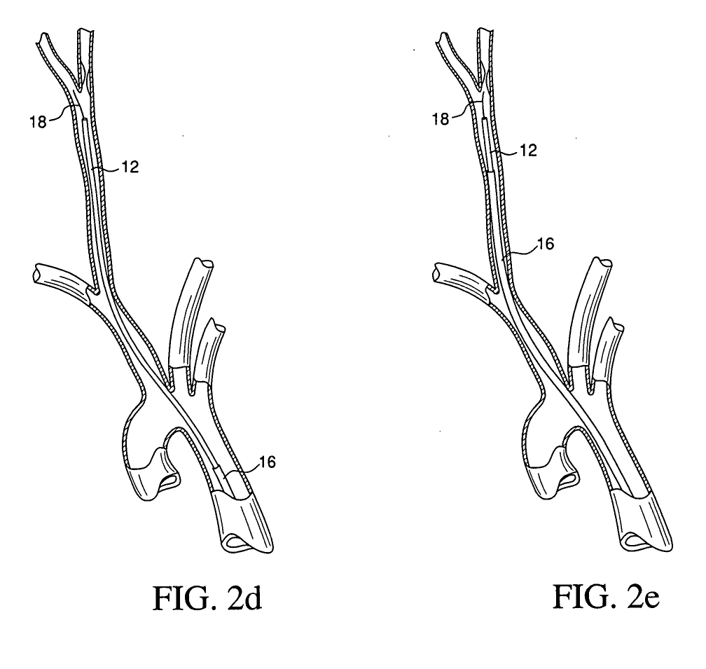

Although the present sheath/catheter system 10 is disclosed below in the form of various contemplated embodiments, the general procedure for implementing the method in accordance with the present invention remains the same. In particular, and with reference to FIGS. 2a to 2e, the procedure is initiated by utilizing the Seldinger technique to place an introducer sheath in the common femoral artery, advancing a guide wire 14 into the ascending aorta to gain access to the aortic arch and advancing a catheter 12 in accordance with the present invention over the guide wire 14. If necessary in the judgment of the doctor performing the procedure, the catheter 12 may be maintained in its stiff/hard state or transformed into a flexible/soft state for insertion over the guide wire 14.

Thereafter, the sheath 16 in accordance with the present invention is advanced into the aorta over the catheter 12 previously placed therein. The sheath 16 is advanced with the sheath 16 in its stiff/hard configuration. The catheter 12 is then positioned for advancement to the carotid artery. A thin or soft guide wire 18 is then inserted into the carotid artery. Thereafter, the sheath 16 and/or catheter 12 are softened and relaxed for increased flexibility.

As those skilled in the art will appreciate, the softening and relaxing of the catheter and/or sheath is based upon the judgment of the doctor performing the procedure. In practice, it is contemplated the determination as to whether and how much the sheath and/or catheter should be softened and relaxed will be balanced based upon the vasculature of the patient, and the relative hardness and flexibility of the catheter and sheath.

With this in mind, and in accordance with a preferred embodiment of the present invention, the catheter 12 is softened and the flexibility is increased, and is then advanced over the guide wire 18 and into the carotid artery. Finally, and in accordance with an optional step, the catheter 12 may be hardened and stiffened once it is positioned in the carotid artery. Thereafter, the sheath 16, in its soft/flexible state, is advanced over the catheter 12 and the guide wire 18 and into the carotid artery. Finally, the sheath 16 is hardened/stiffened.

Once the sheath 16 and catheter 12 are positioned within the carotid artery, an embolic protection device is advanced across the stenotic lesion. Thereafter, the stenotic lesion is pre-dilated using a balloon (if necessary), a stent is deployed across the pre-dilated lesion and the stent is post dilated with a balloon (if necessary). Finally, the embolic protection device is removed.

By utilizing the procedure presented above, the number of steps is reduced for secure placement of a sheath in a particular vessel through a tortuous anatomy. In addition, safer access is provided with reduced trauma to vessels en route to the targeted treatment area and the risk of embolic complications is reduced.

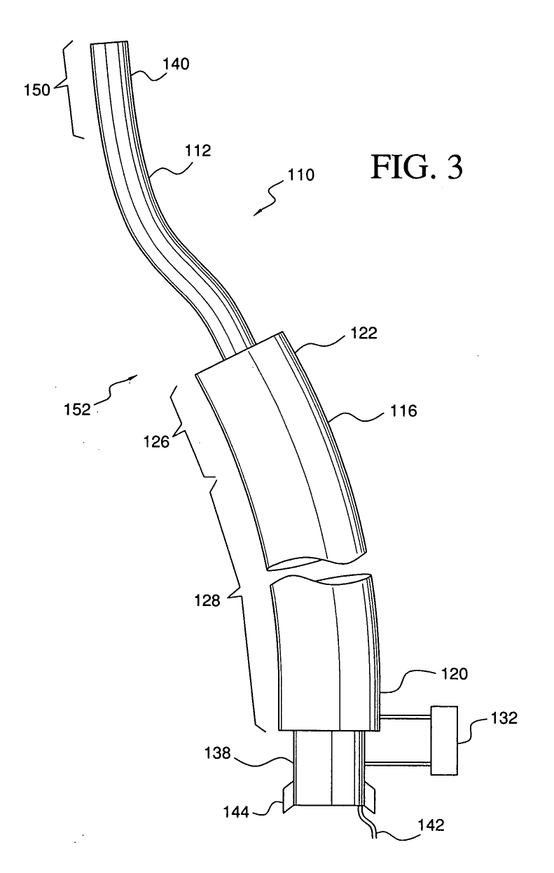

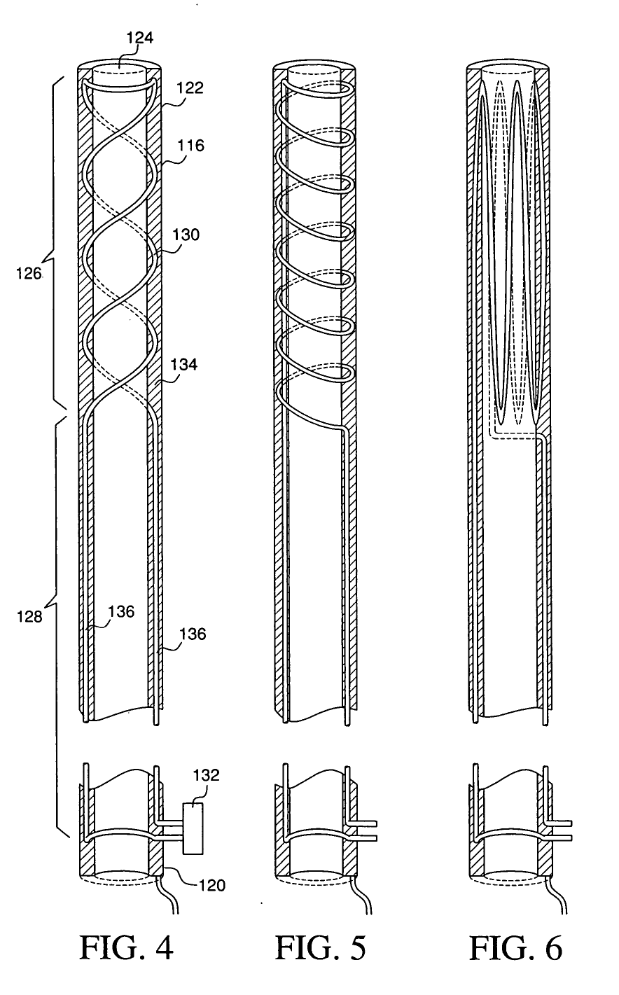

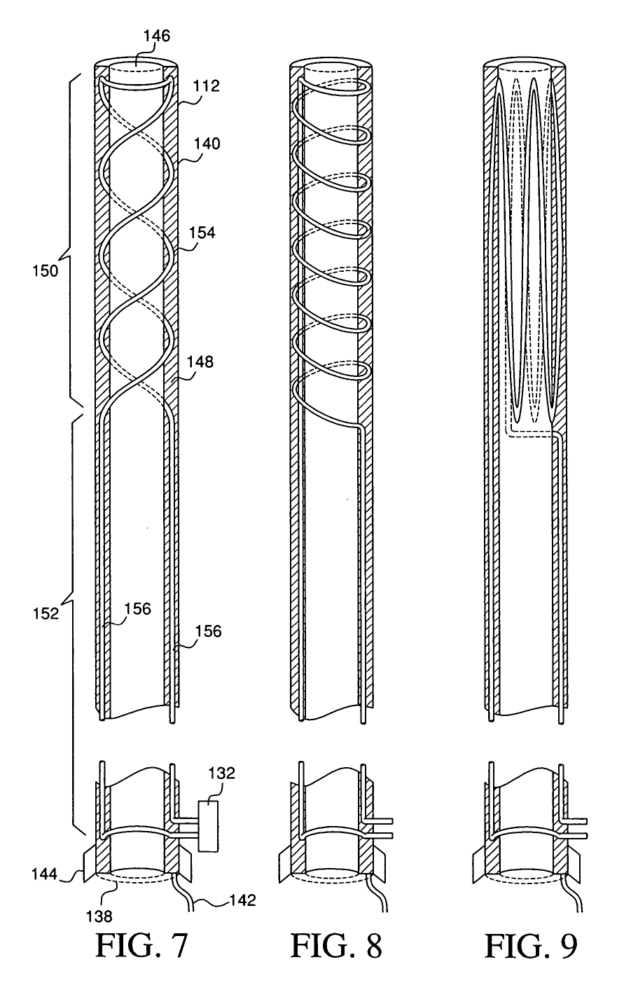

As discussed above, various sheath/catheter designs are contemplated for use in conjunction with the present invention. Referring to FIGS. 3 to 9, a first embodiment of a sheath/catheter system 110 is disclosed. The first embodiment employs a sheath 116 composed of polyolefins (for example, polyethylene), polyurethane, polyamides (for example, nylon), polyether block amides (for example, PEBAX (ELF Atochem)), or polyurethane based shape memory materials. In accordance with a preferred embodiment of the present invention, the wall thickness of the sheath 116 is kept to a minimum in accordance with industry standards (for example, approximately 0.010 to approximately 0.015 inches). The sheath 116 includes a proximal end 120 and a distal end 122, with a central, longitudinally extending lumen 124.

As will be appreciated based upon the following disclosure, the distal end 122 includes a distal tip portion 126 that is selectively controllable with regard to flexibility and hardness. In accordance with a preferred embodiment of the present invention, the distal tip portion 126 constitutes approximately the distal most 3 to 10 cm of the sheath 112. As a result, the sheath may be thought of as being composed of the distal tip portion 126 and the main body portion 128 of the sheath 116.

Controlled stiffness and hardness of the distal tip portion 126 of the sheath 116 is achieved by providing a metal coil(s) 130 embedded within the sheath body 134. The metal coil 130 is secured to an external electrical power source 132 that selectively applies current to the metal coil 130 for heating the metal coil 130 and subsequently heating the material making up the sheath body 134. By heating the metal coil 130 and the distal tip portion 126 of the sheath body 134, the sheath body 134 at distal tip portion 126 will become softer and more flexible. In this way, medical practitioners may selectively control electrical current applied to the metal coil 130 and ultimately the heat generated thereby to control the temperature at the distal tip portion 126 of the sheath 116.

With this in mind, the material of the distal tip portion 126 of the sheath body 134 is specifically designed to offer a controlled hardness and flexibility over a very narrow range and to limit the time it takes to achieve a desired change in softness and flexibility. In accordance with a preferred embodiment of the present invention, the material from which the distal tip portion of the sheath body 134 is composed will undergo a transition from a stiff/hard material to a flexible/soft material when approximately a 3° to 10° Fahrenheit temperature change, more preferably, an approximately a 3° Fahrenheit temperature change, is encountered. In accordance with a preferred embodiment, the present sheath material, and particularly, the distal tip portion 126, is chosen to have a glass transition temperature (T_{g}) of approximately 100° Fahrenheit. As such, at temperatures lower than 100° Fahrenheit, the distal tip portion 126 of the sheath 116 will exhibit traditional stiffness and hardness. However, when the temperature is raised to 100° Fahrenheit, or more, based upon the application of heat via the metal coil 130, the sheath body 134 at the distal tip portion 126 will undergo transition to a more flexible and softer material.

With regard to the hardness of the sheath body 134, the sheath body 134 will range from a hardness of approximately 75 durometer hardness (Shore A) under normal operation conditions and soften to a hardness of approximately 55 durometer hardness (Shore A) upon the application of heat when T_{g} is reached. As mentioned above, the change in hardness is primarily directed to the distal tip portion 126 and the remaining portion (that is, the main body portion 128) of the sheath body 134 may remain at a hardness of approximately 75 durometer hardness (Shore A) while remaining within the spirit of the present invention. Although a preferred hardness range is disclosed in accordance with a preferred embodiment of the present invention, those skilled in the art will appreciate that variations in hardness are certainly possible to suit specific applications without departing from the spirit of the present invention.

As to the orientation of the metal coil 130 within the sheath body 134, it is a preferred embodiment that the metal coil 130 is in the form of a double helix, allowing for consistent application of heat and the completion of an electrical circuit within the sheath body 134. It is further contemplated that improved and controlled application of heat at the distal tip portion 126 of sheath 116 will be achieved by providing only a helical coil design at the distal tip portion 126 and providing straight electrical leads 136 along the main body portion 128 of the sheath 116. However, it is contemplated that other metal coil configurations may be employed without departing from the spirit of the present invention, for example, a proximal to distal coil orientation (that is, a sinusoidal, up and down the catheter configuration as shown in FIG. 6) or a spiral configuration (see FIG. 5) is also contemplated. It is also contemplated the material composition of the metal coil may be varied along the length thereof or the insulation on the coil may be varied along the length thereof, thereby altering the conductivity, and accordingly the resulting heat, being generated upon the application of electrical current.

With regard to the catheter 112, it also includes a proximal end 138 and a distal end 140. The proximal end 138 is provided with traditional ports 142 and coupling members 144 utilized in vascular catheters. The catheter 112 also includes various traditional lumens, for example, a guide wire lumen 146. In accordance with a preferred embodiment of the present invention, the catheter incorporates an “over the wire” guide wire lumen design. Although preferred general structural features of the catheter 112 are disclosed above in accordance with a preferred embodiment of the present invention, those skilled in the art will appreciate that the construction of various catheter components may be varied without departing from the spirit of the present invention.

The catheter body 148 is composed of polyolefins (for example, polyethylene), polyurethane, polyamides (for example, nylon), polyether block amides (for example, PEBAX (ELF Atochem)), or polyurethane based shape memory materials. The catheter will preferably have a wall thickness not exceeding approximately 0.039 inches. The catheter 112 also includes a distal tip portion 150 at the distal end 140 thereof. In accordance with a preferred embodiment of the present invention, the distal tip portion 150 constitutes approximately the distal most 3 to 20 cm of the catheter 112. As a result, the catheter 112 may be thought of as being composed of the distal tip portion 150 and the main body portion 152 of the catheter.

Controlled stiffness and hardness of the catheter body 148 at the distal tip portion 150 is achieved by providing a metal coil(s) 154 embedded within the catheter body 148. The metal coil(s) 154 is secured to an external electrical power source 132 that selectively applies current to the metal coil 154 for heating the metal coil 154 and subsequently heating the material making up the catheter body 148 at the distal tip portion 150. By heating the metal coil 154 and the catheter body 148, the catheter body 148 at the distal tip portion 150 will become softer and more flexible. In this way, medical practitioners may selectively control electrical current applied to the metal coil 154 and the heat generated thereby to ultimately control the temperature of the catheter body 148 at the distal tip portion 150.

With this in mind, the material of the catheter body 148 at the distal tip portion 150 is specifically designed to offer a controlled hardness and flexibility over a very narrow range and to limit the time it takes to achieve a desired change in softness and flexibility. In accordance with a preferred embodiment of the present invention, the material from which the catheter body 148 at the distal tip portion 150 is composed will undergo a transition from a stiff/hard material to a flexible/soft material when approximately a 3 to 10 degree Fahrenheit temperature change, more preferably, approximately a 3 degree Fahrenheit temperature change, is encountered. More particularly, the present catheter material, in particular, the material at the distal tip portion 150, will be chosen to have a T_{g} of approximately 100° Fahrenheit. As such, at temperatures lower than 100° Fahrenheit, the catheter 112 will exhibit traditional stiffness and hardness. However, when the temperature is raised to 100° Fahrenheit, or more, based upon the application of heat via the metal coil 154, the catheter body 148 at the distal tip portion 150 will undergo transition to a more flexible and softer material.

With regard to the hardness of the catheter body 148 the catheter body 148 will range from a hardness of approximately 75 durometer hardness (Shore A) under normal operation conditions and soften to a hardness of approximately 55 durometer hardness (Shore A) upon the application of heat. As mentioned above, the change in hardness is primarily directed to the distal tip portion 150 and the remaining portion of the catheter body 148 may remain at a hardness of approximately 75 durometer hardness (Shore A) while remaining within the spirit of the present invention. Although a preferred hardness range is disclosed in accordance with a preferred embodiment of the present invention, those skilled in the art will appreciate that variations in hardness are certainly possible to suit specific applications without departing from the spirit of the present invention.

As to the orientation of the metal coil 154 within the catheter body 148, it is a preferred embodiment that the metal coil 154 is in the form of a double helix, allowing for consistent application of heat and the completion of an electrical circuit within the catheter body 148. It is further contemplated that improved and controlled application of heat at the distal tip portion 150 of catheter 112 will be achieved by providing only a helical coil design at the distal tip portion 150 and providing straight electrical leads 156 along the main body portion 152 of the catheter 112. However, it is contemplated that other metal coil configurations may be employed without departing from the spirit of the present invention, for example, a proximal distal sinusoidal coil orientation (see FIG. 9) or a spiral configuration (see FIG. 8) are also contemplated. It is also contemplated the material composition of the metal coil may be varied along the length thereof or the insulation on the coil may be varied along the length thereof, thereby altering the conductivity, and accordingly the resulting heat, being generated upon the application of electrical current.

In accordance with a preferred embodiment, the sheath 116 will have a 6 French diameter while the catheter 112 will have a 5 French distal diameter and 6 French proximally to allow smooth transition between the catheter 112 and sheath 116. However, and as those skilled in the art will certainly appreciate, a variety of sizes are contemplated within the spirit of the invention.

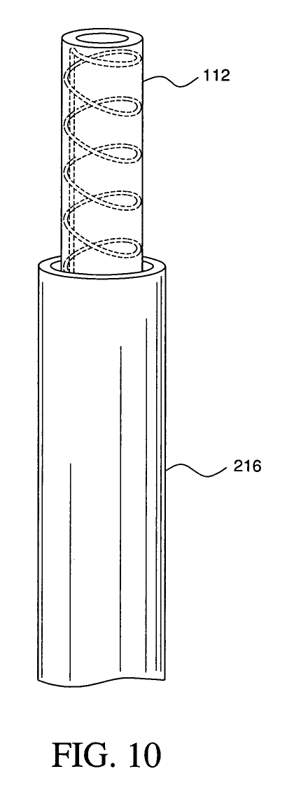

The embodiment disclosed above provides a sheath/catheter system 110 wherein the sheath 116 and catheter 112 both include temperature control systems. However, and with reference to FIG. 10, it is contemplated the sheath 216 may be constructed without a heating mechanism and use the heat generated by the catheter to provide heat for raising temperature of the sheath body for transition between a stiff/hard sheath and a flexible/soft sheath. With this in mind, and with the exception of excluding the metal coil, the construction of the sheath 216 used in accordance with this embodiment will be the same as described above and exhibit a T_{g} and hardness as described above. This embodiment will allow maintaining the wall thickness of the sheath to a minimum, as common in industry standards.

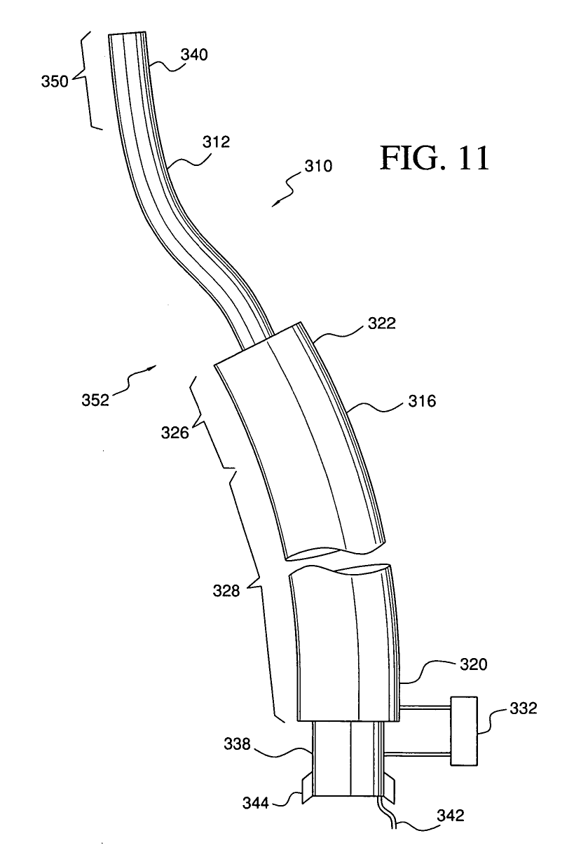

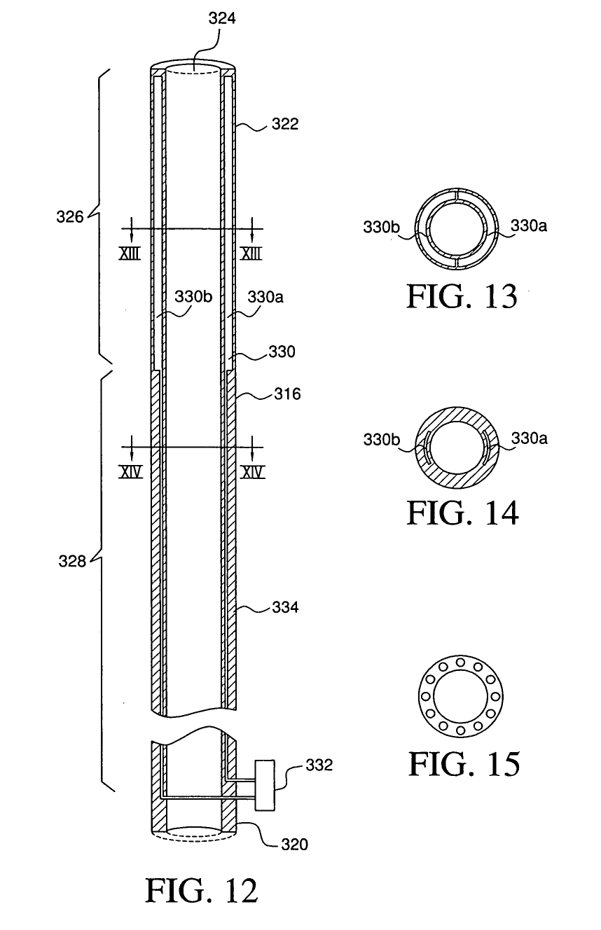

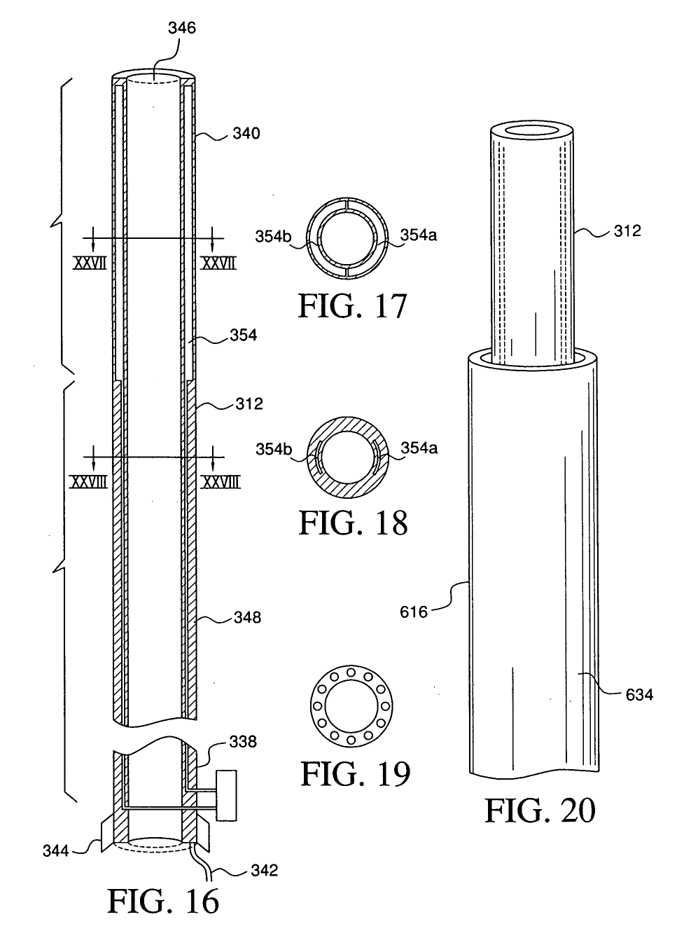

Referring to FIGS. 11 to 19, an alternate embodiment of a sheath/catheter system 310 is disclosed. This alternate embodiment relies upon the flow of a cooling fluid within the catheter 312 and/or sheath 316 to control the hardness and flexibility thereof. As such, the material from which the sheath body 334 and catheter body 348, at least at the respective distal tip portion 326, 350, are constructed preferably exhibits a T_{g} of approximately 98.6° Fahrenheit. With this in mind, the distal tip portions 326, 350 of the catheter 312 and/or sheath 316 will exhibit soft and flexible characteristics when inserted within the body. However, when it is desirable that the catheter 312 and/or sheath 316 exhibit stiffer and harder material characteristics, a cold fluid is flushed through a cooling fluid lumen extending through the catheter 312 and/or sheath 316 via a fluid source. The cold fluid will decrease the temperature below the glass transition temperature, thereby changing the flexibility and hardness thereof as desired by the medical practitioner.

It is contemplated the present embodiment may be varied by using the fluid lumens passing through the catheter and sheath to transport a heating fluid. In accordance with this embodiment, the heating fluid would heat the catheter and sheath in a manner similar to the embodiment described with reference to FIGS. 11 to 19, and the material choice for such an embodiment would be chosen to accommodate the heating, rather than cooling, of the sheath/catheter system.

More particularly, the first embodiment employs a sheath 316 composed of polyolefins (for example, polyethylene), polyurethane, polyamides (for example, nylon), polyether block amides (for example, PEBAX (ELF Atochem)), or polyurethane based shape memory materials. The sheath 316 includes a proximal end 320 and a distal end 322, with a central, longitudinally extending lumen 324. In accordance with a preferred embodiment of the present invention, the wall thickness of the sheath 316 is kept to a minimum in accordance with industry standards (for example, approximately 0.010 to approximately 0.015 inches).

As will be appreciated based upon the following disclosure, the distal end 322 includes a distal tip portion 326 that is selectively controllable with regard to flexibility and hardness. In accordance with a preferred embodiment of the present invention, the distal tip portion 326 constitutes approximately the distal most 3 to 10 cm of the sheath 316. As a result, the sheath 316 may be thought of as being composed of the distal tip portion 326 and the main body portion 328.

Controlled stiffness and hardness of the distal tip portion 326 of the sheath 316 is achieved by providing a cooling fluid lumen 330 within the sheath body 334. The cooling fluid lumen 330 is in fluid communication with a cool fluid source 332 which selectively applies cooling fluid to create a flow of cooling fluid through the cooling fluid lumen 330 and subsequently cooling the material making up the sheath body 334 at the distal tip portion 326. By passing cooling fluid through the cooling fluid lumen 330 and the sheath body 334, the sheath body 334 at the distal tip portion 326 will become harder and stiffer. In this way, medical practitioners may selectively control cooling fluid applied to the cooling fluid lumen 330 and ultimately the sheath body 334 cooling generated thereby to control the temperature of the sheath body 334 at the distal tip portion 326.

With this in mind, the material of the sheath body 334 at the distal tip portion 326 is specifically designed to offer a controlled hardness and flexibility over a very narrow range and to limit the time it takes to achieve a desired change in softness and flexibility. In accordance with a preferred embodiment of the present invention, the material from which the sheath body 334 at the distal tip portion 326 is composed will undergo a transition from a stiff/hard material to a flexible/soft material when approximately a 3 to 10 degree Fahrenheit temperature change, more preferably, approximately a 3 degree temperature change, is encountered. More particularly, the present sheath material at the distal tip portion 326 will be chosen to have a T_{g} of approximately 98.6° Fahrenheit. As such, at temperatures lower than 98.6° Fahrenheit, the sheath 316 at the distal tip portion 326 will exhibit traditional stiffness and hardness. However, when the temperature is raised to 98.6° Fahrenheit, or more, based upon insertion of the sheath 316 within a patient's body, the sheath material at the distal tip portion will undergo transition to a more flexible and softer material. Similarly, when the sheath body 334 at the distal tip portion 326 is cooled to a temperature of less than 98.6° Fahrenheit it returns to its original stiffness and hardness characteristics.

With regard to the hardness of the sheath body 334, the sheath body 334 will range from a hardness of approximately 75 durometer hardness (Shore A) under normal operation conditions and soften to a hardness of approximately 55 durometer hardness (Shore A) upon insertion into the body of the patient. As mentioned above, the change is hardness is primarily directed to the distal tip portion 326 and the remaining portion of the sheath body 334 may remain at a hardness of approximately 75 durometer hardness (Shore A) while remaining within the spirit of the present invention. Although a preferred hardness range is disclosed in accordance with a preferred embodiment of the present invention, those skilled in the art will appreciate that variations in hardness are certainly possible to suit specific applications without departing from the spirit of the present invention.

As to the orientation of the cooling fluid lumen 330 within the sheath body 334, it is a preferred embodiment that the cooling fluid lumen 330 is shaped and dimensioned to apply cooling to the distal tip portion 326 in a controlled and consistent manner. With this in mind, it is contemplated the cooling fluid lumen 330 will be in the form of multiple lumen portions 330a, 330b extending along the length of the sheath body 334 in a manner allowing for the flow of cooling fluid from the proximal end 320 of the sheath 316, to the distal end 322 of the sheath 316 and back to the proximal end 320 of the sheath 316. In order to control the application of cooling effect to the distal tip portion 326, the lumen portions 330a, 330b are enlarged within the distal tip portion 326 of the sheath 316 providing for an improved application of cooling effect in this area. In addition to this lumen design, it is also contemplated that helical, sinusoidal or other lumen designs (for example, multiple lumens as shown in FIG. 15) may be employed without departing from the spirit of the present invention.

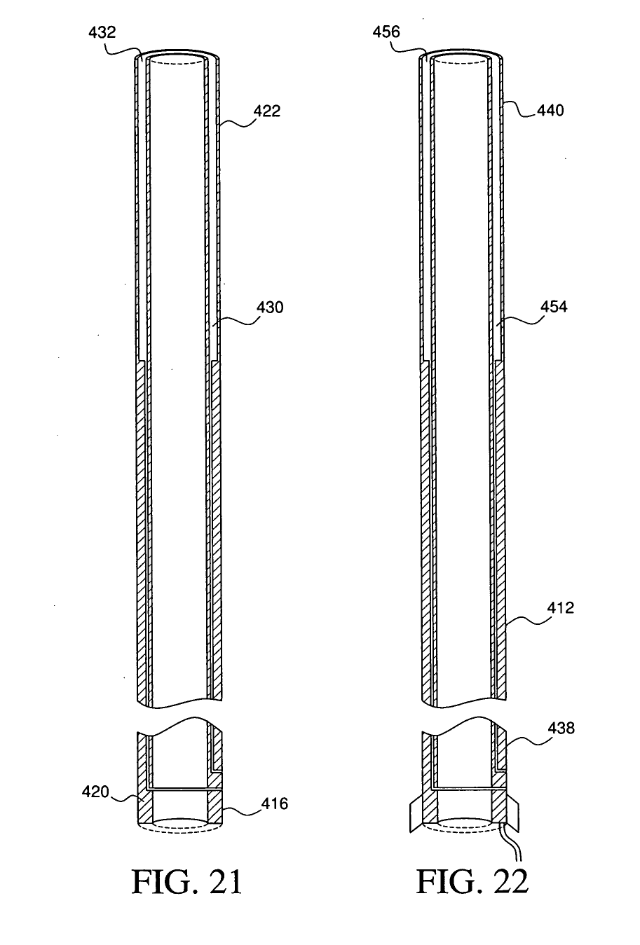

Referring to FIG. 21, an alternate fluid lumen 430 is shown. In accordance with this embodiment, fluid is pumped in through the proximal end 420 of the sheath 416, passes through the cooling fluid lumen 430 and exits through a port 432 in the distal end 422 thereof As those skilled in the art will certainly appreciate the cooling fluid used in such a system would necessarily need to be biocompatible.

With regard to the catheter 312, it also includes a proximal end 338 and a distal end 340. The proximal end 338 is provided with traditional ports 342 and coupling members 344 utilized in vascular catheters. The catheter body 348 is composed of polyolefins (for example, polyethylene), polyurethane, polyamides (for example, nylon), polyether block amides (for example, PEBAX (ELF Atochem)), or polyurethane based shape memory materials, and has a wall thickness which preferably does not exceed approximately 0.039 inches in accordance with a preferred embodiment of the present invention. The catheter 312 also includes traditional lumens, for example, a guide wire lumen 146. In accordance with a preferred embodiment of the present invention, the catheter 312 incorporates an “over the wire” guide wire lumen 346 design. Although preferred general structural features of the catheter 312 are disclosed above in accordance with a preferred embodiment of the present invention, those skilled in the art will appreciate that the construction of various catheter components may be varied without departing from the spirit of the present invention.

As will be appreciated based upon the following disclosure, the distal end 340 includes a distal tip portion 350 that is selectively controllable with regard to flexibility and hardness. In accordance with a preferred embodiment of the present invention, the distal tip portion 350 constitutes approximately the distal most 3 to 20 cm of the catheter 312. As a result, the catheter 312 may be thought of as being composed of the distal tip portion 350 and the main body portion 352 of the catheter 312.

Controlled stiffness and hardness of the catheter 312 at the distal tip portion 350 is achieved by providing a cooling fluid lumen 354 within the catheter body 348. The cooling fluid lumen 354 is in fluid communication with a cool fluid source 332 that selectively applies cooling fluid to create a flow of cooling fluid through the cooling fluid lumen 354 and subsequently cooling the material making up the catheter body 348 at the distal tip portion 350. By passing cooling fluid through the cooling fluid lumen 354 and the catheter body 348, the catheter body 348 at the distal tip portion 350 will become harder and stiffer. In this way, medical practitioners may selectively control cooling fluid applied to the cooling lumen and ultimately the catheter body 348 cooling generated thereby to ultimately control the temperature of the catheter body 348 at the distal tip portion 350.

With this in mind, the material of the catheter body 348 at the distal tip portion 350 is specifically designed to offer a controlled hardness and flexibility over a very narrow range and to limit the time it takes to achieve a desired change in softness and flexibility. In accordance with a preferred embodiment of the present invention, the material from which the catheter body 348 at the distal tip portion 350 is composed will undergo a transition from a stiff/hard material to a flexible/soft material when approximately a 3 to 10 degree Fahrenheit temperature change, more preferably, approximately a 3 degree Fahrenheit temperature change, is encountered. More particularly, the present catheter material at the distal tip portion 350 is chosen to have a glass transition temperature T_{g} of approximately 98.6° Fahrenheit. As such, at temperatures lower than 98.6° Fahrenheit, the catheter 312 at the distal tip portion 350 will exhibit traditional stiffness and hardness. However, when the temperature is raised to 98.6° Fahrenheit, or more, based upon insertion of the catheter 312 within a patient's body, the catheter material at the distal tip portion 350 will undergo transition to a more flexible and softer material. Similarly, when the catheter body 348 at the distal tip portion 350 is cooled to a temperature of less than 98.6° Fahrenheit it returns to its original stiffness and hardness characteristics.

With regard to the hardness of the catheter body 348, the catheter body 348 will range from a hardness of approximately 75 durometer hardness (Shore A) under normal operation conditions and soften to a hardness of approximately 55 durometer hardness (Shore A) upon insertion into the body of the patient. As mentioned above, the change in hardness is primarily directed to the distal tip portion 350 and the remaining portion of the catheter body 348 may remain at a hardness of approximately 75 durometer hardness (Shore A) while remaining within the spirit of the present invention. Although a preferred hardness range is disclosed in accordance with a preferred embodiment of the present invention, those skilled in the art will appreciate that variations in hardness are certainly possible to suit specific applications without departing from the spirit of the present invention.

As to the orientation of the cooling fluid lumen 354 within the catheter body 348, it is a preferred embodiment that the cooling fluid lumen 354 is shaped and dimensioned to apply cooling to the distal tip portion 350 in a controlled and consistent manner. With this in mind, it is contemplated the cooling fluid lumen 354 will be in the form of multiple lumen portions 354a, 354b extending along the length of the catheter body 348 in a manner allowing for the flow of cooling fluid from the proximal end 338 of the catheter 312, to the distal end 340 of the catheter 312 and back to the proximal end of 338 the catheter 312. In order to control the application of cooling effect to the distal tip portion 350, the lumen portions 354a, 354b are enlarged within the distal tip portion 350 of the catheter 312 providing for an improved application of cooling effect in this area. In addition to this lumen design, it is also contemplated that helical, sinusoidal or other lumen designs (for example, a multiple lumen design as shown in FIG. 19) may be employed without departing from the spirit of the present invention.

Referring to FIG. 22, an alternate fluid lumen 454 is shown. In accordance with this embodiment, fluid is pumped in through the proximal end 438 of the catheter 412, passes through the cooling fluid lumen 454 and exits through a port 456 in the distal end 440 thereof. As those skilled in the art will certainly appreciate the cooling fluid used in such a system would necessarily need to be biocompatible.

In accordance with a preferred embodiment, the sheath 316 will have a 6 French diameter while the catheter 312 will have a 5 French distal diameter and 6 French proximally to allow smooth transition between the catheter 312 and sheath 316. However, and as those skilled in the art will certainly appreciate, a variety of sizes are contemplated within the spirit of the invention.

The embodiment disclosed above provides a sheath/catheter system 310 wherein the sheath 316 and catheter 312 both include temperature control systems. However, and with reference to FIG. 20, it is contemplated the sheath 616 may be constructed without a cooling mechanism and use the cooling effect generated by the catheter 312 to provide for lowering the temperature of the sheath body 634 for transition between a stiff/hard sheath 616 and a flexible/soft sheath 616. With this in mind, and with the exception of excluding the fluid lumen, the construction of the sheath 616 used in accordance with this embodiment will be the same as described above and exhibit a T_{g} and hardness as described above.

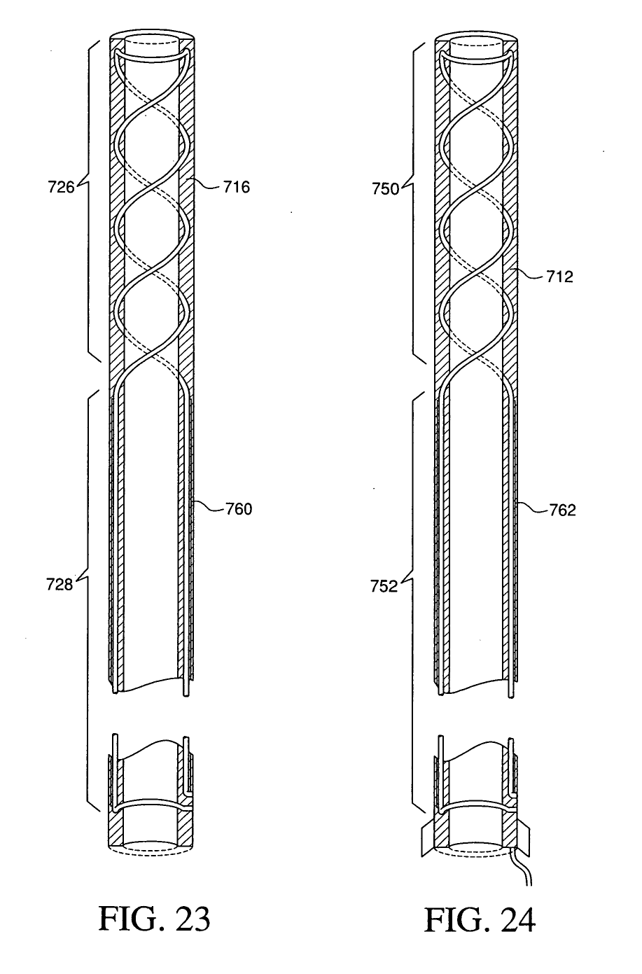

As discussed above, it is preferred that only the distal tip portion of the catheter or sheath be provided with the ability to change flexibility/hardness. With this in mind, it is further contemplated that either the metal coil heated catheter or the cold fluid cooled catheter may be provided with insulation to prevent undesirable interaction between the temperature changes in the catheter and the temperature of the body in which the catheter is passing. As shown in FIGS. 23 and 24, the insulation 760, 762 will preferably extend along the length of the catheter 712 and/or sheath 716 only along the main body portion 728, 752 and the distal tip portion 726, 750 will be left uninsulated such that it responds to heating or cooling in the desired manner. In this way, the insulation 760, 762 is used to control the temperature changes within the main body portions 728, 752 of the catheter 712 and/or sheath 716 so that flexibility/hardness is only altered at the distal tip portion 726, 752.

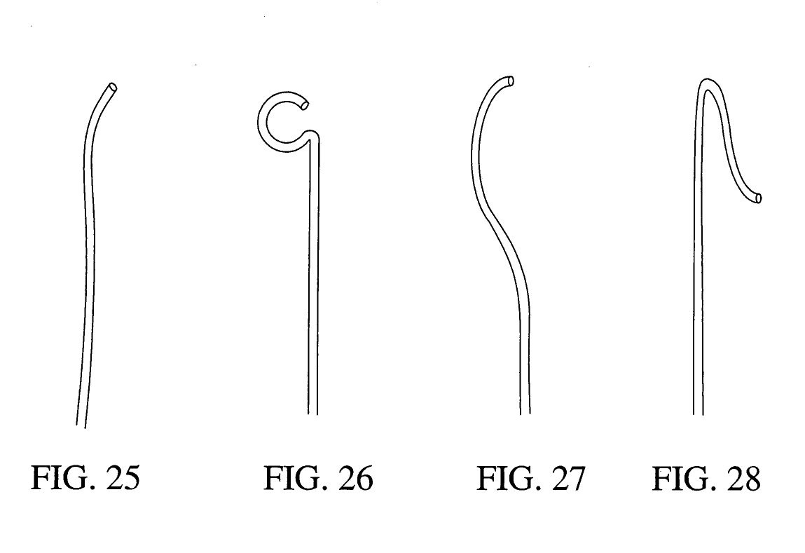

In accordance with a preferred embodiment, various catheter tip designs are contemplated for use. These tip designs are disclosed in FIGS. 25 to 28, although those skilled in the art will appreciate that other tip designs may be employed without departing from the spirit of the present invention.

It is further contemplated that the distal tip portion of the catheter and/or sheath may be composed of materials different from the main body portion of the catheter and/or sheath in order to provide controlled flexibility at the various points along the body of the catheter and/or sheath.

In accordance with alternate embodiments, it is contemplated that electric, mechanical or UV actuation mechanisms may be employed in the conversion of the present sheath/catheter system. For example, an electrical system employing a Nitinol-based structure, for example, a cage or lattice, in a plastic tubing wall may be employed. Similarly, the sheath/catheter system may be constructed of shape memory polymers that change flexibility and hardness upon the application of electrical current. Also, a mechanical telescoping effect, for example, pushing a discrete liner of greater stiffness into the placed tube to increase its stiffness, may be employed. Finally, UV energy may be utilized to alter the characteristics of the sheath/catheter system, for example, by constructing the sheath/catheter system of shape memory polymers that change flexibility and hardness upon the application of optical energy.

As those skilled in the art will readily appreciate, various mechanisms for heating and cooling a sheath/catheter system to control hardness and flexibility have been described above, and it is contemplated that these mechanisms may be employed in various combinations while remaining within the spirit of the present invention.

While the preferred embodiments have been shown and described, it will be understood that there is no intent to limit the invention by such disclosure, but rather, is intended to cover all modifications and alternate constructions falling within the spirit and scope of the invention.

1. A sheath/catheter system, comprising:
a catheter including a proximal end and a distal end, the distal end including a distal tip portion exhibiting controlled hardness and flexibility; and
a sheath including a proximal end and a distal end; and
means for controlling the hardness and flexibility of the the distal tip portion of the catheter.

2. The sheath/catheter system according to claim 1, wherein the distal end of the sheath includes a distal tip portion exhibiting controlled hardness and flexibility, and the means for controlling also controls the hardness and flexibility of the distal tip portion of the sheath.

3. The sheath/catheter system according to claim 1, wherein the means for controlling the hardness and flexibility of the distal tip portion of the catheter includes a metal coil formed in the catheter.

4. The sheath/catheter system according to claim 3, wherein the means for controlling the hardness and flexibility of the distal tip portion of the sheath is the metal coil formed in the catheter.

5. The sheath/catheter system according to claim 3, wherein the means for controlling the hardness and flexibility of the distal tip portion of the sheath includes a metal coil formed in the sheath.

6. The sheath/catheter system according to claim 3, wherein the metal coil is connected to a source of electricity.

7. The sheath/catheter system according to claim 3, wherein the metal coil has a helical shape.

8. The sheath/catheter system according to claim 1, wherein the means for controlling the hardness and flexibility of the distal tip portion of the catheter includes a fluid lumen coupled to a fluid source which passes through the catheter.

9. The sheath/catheter system according to claim 8, wherein the means for controlling the hardness and flexibility of the distal tip portion of the sheath is the fluid lumen coupled to the fluid source formed in the catheter.

10. The sheath/catheter system according to claim 8, wherein the means for controlling the hardness and flexibility of the distal tip portion of the sheath includes a fluid lumen coupled to a fluid source which passes through the catheter.

11. The sheath/catheter system according to claim 8, wherein the fluid source is cool fluid source.

12. The sheath/catheter system according to claim 8, wherein the fluid source is hot fluid source.

13. The sheath/catheter system according to claim 2, wherein the distal tip portion of the catheter is approximately the distal most 3 cm to 20 cm of the catheter.

14. The sheath/catheter system according to claim 2, wherein the distal tip portion of the sheath is approximately the distal most 3 cm to 10 cm of the sheath.

15. A method for vascular access, comprising:
introducing a catheter into a vessel;
advancing a sheath over the catheter and into the vessel;
altering the catheter or sheath to change the respective flexibility and hardness thereof;
further advancing the catheter or sheath within the vascular system to a predetermined location.

16. The method according to claim 15, further including advancing the catheter and sheath into a carotid artery.

17. The method according to claim 15, wherein the step of altering includes altering both the catheter and sheath to change the respective hardness and flexibility thereof.

18. The method according to claim 17, wherein both the catheter and sheath are altered to increase softness and flexibility.

19. The method according to claim 15, wherein both the catheter and sheath are altered to increase softness and flexibility.

20. The method according to claim 15, further including the step of altering the catheter or sheath to increase stiffness and hardness upon advancement of the catheter or sheath to the predetermined location.
